(19) United States
(12) Patent Application Publication
(10) Pub. No.: US 2006/0085922 A1
(43) Pub. Date: Apr. 27, 2006

(54) Oxidizing composition comprising at least one electrophilic monomer and at least one non-benzoquinone oxidant

(76) Inventor: Gregory Plos, Tokyo (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/248,149

(22) Filed: Oct. 13, 2005

Related U.S. Application Data
(60) Provisional application No. 60/622,624, filed on Oct. 28, 2004.

(30) Foreign Application Priority Data
Oct. 13, 2004 (FR) 0410804

(51) Int. Cl. A61K 8/00

(52) U.S. Cl. 8/405

(57) ABSTRACT

Disclosed herein is an oxidizing composition for the bleaching and/or permanent reshaping of keratin fibers, comprising at least one electrophilic monomer and at least one non-benzoquinone oxidant. Also disclosed herein is a method for bleaching and/or permanently reshaping keratin fibers comprising applying to the keratin fibers an oxidizing composition comprising at least one electrophilic monomer and at least one non-benzoquinone oxidant.

This application claims benefit of U.S. Provisional Application No. 60/622,624, filed Oct. 28, 2004, the contents of which are incorporated herein by reference. This application also claims benefit of priority under 35 U.S.C. § 119 to French Patent Application No. 0410804, filed Oct. 13, 2004, the contents of which are also incorporated herein by reference.

Disclosed herein is an oxidizing composition for the bleaching and/or permanent reshaping of keratin fibers such as the hair, comprising, in a cosmetically acceptable medium, at least one electrophilic monomer and at least one non-benzoquinone oxidant.

It is known to bleach keratin fibers, for example, human keratin fibers such as the hair, with bleaching compositions containing at least one oxidant and at least one alkaline compound such as amines and alkali metal silicates.

The processes of permanent reshaping (“perming”) of keratin fibers that are known from the prior art comprise, generally, two steps. In the first step the keratin fibers are contacted with a reducing composition so as to reduce the disulphide bridges these fibers contain. Before, after, or during the reduction of these disulphide bridges, the hair is shaped as desired. The second step is a fixing step required to reform the disulphide bridges and thus stabilize the shape imparted. This operation is commonly performed using compositions comprising at least one oxidant.

The oxidants conventionally used for dyeing or for the fixing step in a keratin fiber perming process include hydrogen peroxide and compounds capable of producing hydrogen peroxide by hydrolysis, such as urea peroxide, and also persalts such as perborates, percarbonates, and persulphates. Hydrogen peroxide and persulphates are the most commonly used oxidants.

The use of such oxidants has the drawback of degrading the keratin fibers and adversely affecting their cosmetic properties, leading, for example, to difficulty in disentangling, an unpleasant feel, and/or hair which is rough and lifeless.

French Patent Application No. 2 833 489 discloses a composition for treating keratin fibers which comprises electrophilic monomers. A composition of this kind allows hair to be obtained which is ideally coated and not greasy.

The aim of the present disclosure is to provide oxidizing compositions which do not exhibit the drawbacks of the known prior art products. For example, the present disclosure is directed to oxidizing compositions for the bleaching of keratin fibers, which make it possible to obtain a lightening effect on the keratin fibers without degrading them and without adversely affecting their cosmetic properties. The present disclosure is also directed to oxidizing compositions for the permanent reshaping of keratin fibers, which make it possible to produce permanent reshaping of the keratin fibers without degrading them or adversely affecting their cosmetic properties.

Thus, disclosed herein is an oxidizing composition comprising, in a cosmetically acceptable medium, at least one electrophilic monomer and at least one non-benzoquinone oxidant. When the composition in accordance with the present disclosure is used for bleaching keratin fibers it makes it possible to obtain a lightening effect on the fibers without degrading them and without adversely affecting their cosmetic properties. When the composition in accordance with the present disclosure is used for the permanent reshaping of keratin fibers it makes it possible to obtain a permanent reshaping of these fibers without degrading them and without adversely affecting their cosmetic properties.

Also disclosed herein is a method for bleaching and/or permanently reshaping keratin fibers comprising applying to the keratin fibers an oxidizing composition comprising, in a cosmetically acceptable medium, at least one electrophilic monomer and at least one oxidant. Further disclosed herein is a multi-compartment device for the implementation of this method.

Oxidant

According to at least one embodiment of the present disclosure, the at least one oxidant may be chosen from, for example:
hydrogen peroxide and agents capable of producing hydrogen peroxide by hydrolysis, such as urea peroxide;
alkali metal or ammonium persulphates, perborates and percarbonates;
alkali metal bromates;
magnesium monoperoxyphthalate;
nitrites, such as alkali metal, alkaline earth metal, and ammonium nitrites, for example, sodium nitrite and ammonium nitrite, and organic nitrite derivatives;
periodic acid and its water-soluble salts, such as the lithium, sodium, potassium, rubidium, caesium, magnesium, calcium, strontium, manganese, iron, copper, zinc, and aluminium salts. In one embodiment, the salts are sodium and potassium salts;
alkali metal ferricyanides, for example, potassium ferricyanide;
silver oxide;
Fenton's reagent;
lead(IV) oxide;
metal anions selected from permanganates and dichromates, for example, potassium permanganate and sodium dichromate;
metal salts from groups III to VIII of the Periodic Table, such as manganese, cobalt, iron, copper, and silver salts, for instance, manganese sulphate, manganese lactate, cobalt chloride, ferric chloride, cupric chloride, ammoniacal silver nitrate, and copper salts. These metal salts may be in the free state or adsorbed;
rare earth salts selected from the salts of lanthanides, salts of cerium Ce³⁺ or Ce⁴⁺, salts of lanthanum La³⁺, salts of europium Eu²⁺ or Eu³⁺, salts of gadolinium Gd³⁺, salts of ytterbium Yb²⁺ or Yb³⁺, and salts of dysprosium Dy³⁺, for example, sulphates, chlorides, and nitrates. In one embodiment, the rare earth salts are selected from sulphate and chloride salts of cerium Ce³⁺ or Ce⁴⁺; and
sodium hypochlorite.

According to at least one embodiment of the present disclosure, the at least one oxidant may be chosen from hydrogen peroxide and agents capable of producing hydrogen peroxide by hydrolysis, such as urea peroxide.

According to another embodiment of the present disclosure the at least one oxidant may be chosen from persulphates.

The at least one oxidant may be present in the composition in an amount ranging from 0.05% to 40% by weight, for example, from 0.1% to 35% by weight, or from 0.25% to 25% by weight relative to the total weight of the composition.

Electrophilic Monomer

In at least one embodiment of the present disclosure the at least one electrophilic monomer may be chosen from compounds of formula (I):

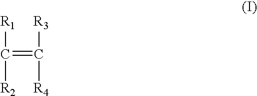

in which:

R₁ and R₂, are, independently of one another, minimally or non-electron-withdrawing groups (minimally or non-inductively-withdrawing groups) such as:

hydrogen,
saturated or unsaturated, linear, branched, or cyclic hydrocarbon-based groups comprising, for example, from 1 to 20, or from 1 to 10 carbon atoms and optionally comprising at least one atom chosen from nitrogen, oxygen, and sulphur, and optionally substituted with at least one group chosen from —OR, —COOR, —COR, —SH, —SR, —OH, and halogen atoms,
modified or unmodified polyorganosiloxane residues, and
polyoxyalkylene groups;
   R₃ and R₄ are, independently of one another, electron-withdrawing (or inductively withdrawing) group chosen for example, from —N(R)₃⁺, —S(R)₂⁺, —SH₂⁺, —NH₃⁺, —NO₂, —SO₂R, —C≡N, —COOH, —COOR, —COSR, —CONH₂, —CONHR, —F, —Cl, —Br, —I, —OR, —COR, —SH, —SR, and —OH groups, linear or branched alkenyl groups, linear or branched alkynyl groups, C₁-C₄ mono- or polyfluoroalkyl groups, aryl groups such as phenyl, and aryloxy groups such as phenyloxy; and
   R is chosen from saturated or unsaturated linear, branched, or cyclic hydrocarbon-based groups comprising, for example, from 1 to 20, or from 1 to 10 carbon atoms, and optionally comprising at least one atom chosen from nitrogen, oxygen, and sulphur, and optionally substituted by at least one group chosen from —OR′, —COOR′, —COR′, —SH, —SR′, and —OH, halogen atoms, and polymer residues obtainable by free-radical polymerization, condensation, or ring opening, wherein R′ is chosen from C₁-C₁₀ alkyl radicals.

As used herein, an electron-withdrawing or inductively-withdrawing (−I) group is any group which is more electronegative than carbon. Reference may be made, for example, to P. R. Wells, Prog. Phys. Org. Chem., vol. 6, page 111 (1968).

As used herein, a minimally or non-electron-withdrawing group is any group whose electronegativity is less than or equal to that of carbon.

In at least one embodiment of the present disclosure, the alkenyl and/or alkynyl groups of R₃ and R₄ in formula (I) may comprise from 2 to 20 carbon atoms, for example, from 2 to 10 carbon atoms.

Non-limiting examples of saturated or unsaturated linear, branched, or cyclic hydrocarbon-based groups comprising from 1 to 20 carbon atoms, or from 1 to 10 carbon atoms, include, for example, linear or branched alkyl groups, linear or branched alkenyl groups, and linear or branched alkynyl groups, such as methyl, ethyl, n-butyl, tert-butyl, isobutyl, pentyl, hexyl, octyl, butenyl and butynyl groups; cycloalkyl groups; and aromatic groups.

Examples of substituted hydrocarbon-based groups include, for example, hydroxyalkyl and polyhaloalkyl groups.

Examples of unmodified polyorganosiloxanes include, but are not limited to, polyalkylsiloxanes such as polydimethylsiloxanes, polyarylsiloxanes such as polyphenylsiloxanes, and polyarylalkylsiloxanes such as polymethylphenylsiloxanes.

Suitable modified polyorganosiloxanes include, for example, polydimethylsiloxanes comprising polyoxyalkylene, siloxy, silanol, amine, imine, and/or fluoroalkyl groups.

Non-limiting examples of polyoxyalkylene groups include polyoxyethylene groups and polyoxypropylene groups comprising, for example, from 1 to 200 oxyalkylene units.

Examples of mono- or polyfluoroalkyl groups include, for example, groups such as —(CH₂)ₙ—(CF₂)ₘ—CF₃ and —(CH₂)ₙ—(CF₂)ₘ—CHF₂, wherein n is a number ranging from 1 to 20 and m is a number ranging from 1 to 20.

In accordance with at least one embodiment of the present disclosure, the substituents R₁ to R₄ may optionally be substituted with at least one group having cosmetic activity. Cosmetic activities may be obtained, for example, from groups comprising at least one dye, antioxidant, UV filter, and/or conditioning function.

Groups comprising a dye function include, but are not limited to, azo, quinone, methine, cyanomethine, and triarylmethane groups.

Examples of groups comprising an antioxidant function include, for example, butylated hydroxyanisole (BHA), butylated hydroxytoluene (BHT), and vitamin E groups.

Non-limiting examples of groups comprising a UV filter function include benzophenone, cinnamate, benzoate, benzylidenecamphor, and dibenzoylmethane groups.

Suitable groups comprising a conditioning function include, for example, cationic groups and groups of fatty ester type.

In at least one embodiment of the present disclosure, the at least one electrophilic monomer may, for example, be chosen from:
benzylidenemalononitrile (A), 2-(4-chlorobenzylidene)malononitrile (A1), ethyl 2-cyano-3-phenylacrylate (B), and ethyl 2-cyano-3-(4-chlorophenyl) acrylate (B1), described, for example, in Sayyah, J. Polymer Research, p. 97 (2000): 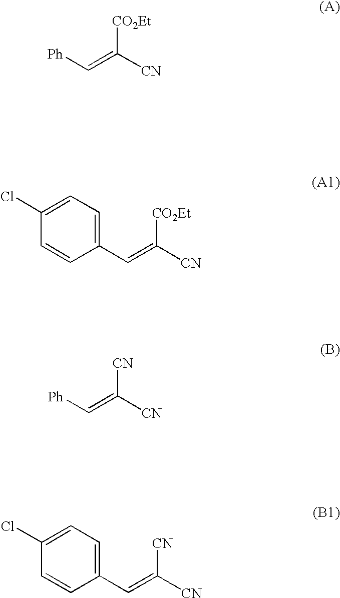
methylidenemalonate derivatives such as diethyl 2-methylenemalonate (C), described, for example, in Hopff, Makromolekulare Chemie, p. 95 (1961); De Keyser, J. Pharm. Sci., p. 67 (1991), and Klemarczyk, Polymer, p. 173 (1998): 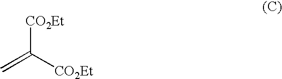
ethyl 2-ethoxycarbonylmethyleneoxycarbonylacrylate (D), described, for example, in Breton, Biomaterials, p. 271 (1998) and Couvreur, Pharmaceutical Research, p. 1270 (1994): 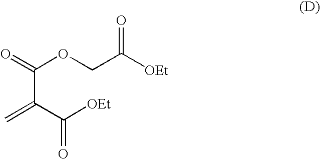
methyl α-(methylsulphonyl)acrylate derivatives (K), ethyl α-(methylsulphonyl)acrylate derivatives (L), methyl α-(tert-butylsulphonyl)acrylate derivatives (M), tert-butyl α-(methylsulphonyl)acrylate derivatives (N), and tert-butyl α-(tert-butylsulphonyl)acrylate derivatives (O), described, for example, in Gipstein, J. Org. Chem., 1980, p. 1486, 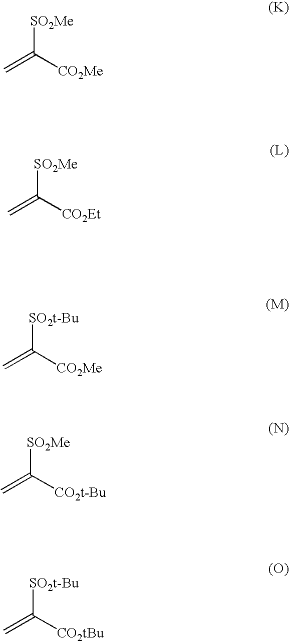
1,1-bis(methylsulphonyl)ethylene derivatives (P), 1-acetyl-1-methylsulphonylethylene derivatives (Q), methyl α-(methylsulphonyl)vinylsulfonate derivatives (R), and a-methylsulphonylacrylonitrile derivatives (S), described, for example, in U.S. Pat. No. 2,748,050:

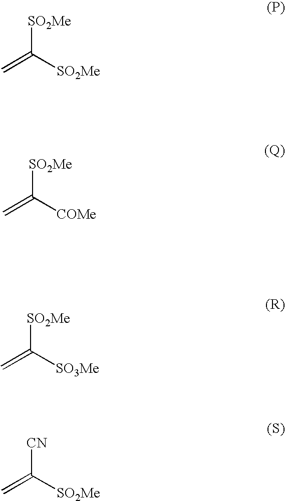

According to at least one embodiment of the present disclosure, the at least one electrophilic monomer may be selected from cyanoacrylates of formula (II): 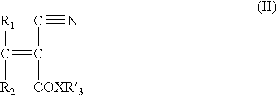
in which:
X is chosen from NH, S, and O;
R₁ and R₂, are, independently of one another, minimally or non-electron-withdrawing groups (minimally or non-inductively-withdrawing groups) such as:
hydrogen,
saturated or unsaturated, linear, branched, or cyclic hydrocarbon-based groups comprising, for example, from 1 to 20, or from 1 to 10 carbon atoms and optionally comprising at least one atom chosen from nitrogen, oxygen, and sulphur, and optionally substituted with at least one group chosen from —OR, —COOR, —COR, —SH, —SR, —OH, and halogen atoms,
modified or unmodified polyorganosiloxane residues, and
polyoxyalkylene groups;
   R′₃ is chosen from hydrogen and the radical R;
   R is chosen from saturated or unsaturated linear, branched, or cyclic hydrocarbon-based groups comprising, for example, from 1 to 20, or from 1 to 10 carbon atoms, and optionally comprising at least one atom chosen from nitrogen, oxygen, and sulphur, and optionally substituted by at least one group chosen from —OR′, —COOR′, —COR′, —SH, —SR′, and —OH, halogen atoms, and polymer residues obtainable by free-radical polymerization, condensation, or ring opening; and
   R′ is chosen from C₁-C₁₀ alkyl radicals.

In at least one embodiment, in formulae (I) and (II), R₁ and R₂ may represent hydrogen.

In another embodiment, in formula (II), X may be O and R′₃ may be chosen from C₆-C₁₀ alkyl radicals.

Compounds of formula (II) may include, for example, the following monomers:

a) monomers belonging to the polyfluoroalkyl 2-cyanoacrylate class, such as the 2,2,3,3-tetrafluoropropyl ester of 2-cyano-2-propenoic acid, of formula (III): 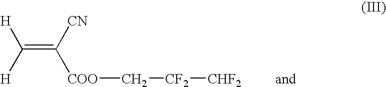
and the 2,2,2-trifluoroethyl ester of 2-cyano-2-propenoic acid, of formula (IV): 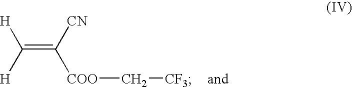
and

b) monomers belonging to the alkyl or alkoxyalkyl 2-cyanoacrylate class, of formula (V): 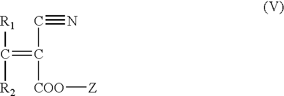
in which:
Z is chosen from C₁-C₁₀ alkyl radicals and C₁-C₄ alkoxy-C₁-C₁₀ alkyl radicals; and
R₁ and R₂, are, independently of one another, minimally or non-electron-withdrawing groups (minimally or non-inductively-withdrawing groups) such as:
hydrogen,
saturated or unsaturated, linear, branched, or cyclic hydrocarbon-based groups comprising, for example, from 1 to 20, or from 1 to 10 carbon atoms and optionally comprising at least one atom chosen from nitrogen, oxygen, and sulphur, and optionally substituted with at least one group chosen from —OR, —COOR, —COR, —SH, —SR, —OH, and halogen atoms,
modified or unmodified polyorganosiloxane residues, and polyoxyalkylene groups.

In one embodiment of the present disclosure, in formula (V), Z may be chosen from C₆-C₁₀ alkyl radicals and R₁ and R₂ may represent hydrogen.

In at least one embodiment of the present disclosure, the at least one electrophilic monomer may be chosen, for example, from ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate, and isoamyl 2-cyanoacrylate.

In another embodiment of the present disclosure, the at least one electrophilic monomer is chosen from monomers belonging to the alkyl or alkoxyalkyl 2-cyanoacrylate class of formula (V).

In a further embodiment, the at least one electrophilic monomer may be chosen from compounds of formula (VI) and mixtures thereof: 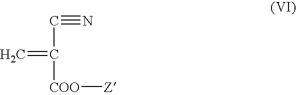
in which Z′ is selected from —(CH₂)₇—CH₃; —CH(CH₃)—(CH₂)₅—CH₃; —CH₂—CH(C₂H₅)—(CH₂)₃—CH₃; —(CH₂)₅—CH(CH₃)—CH₃; and —(CH₂)₄—CH(C₂H₅)—CH₃ radicals.

The at least one electrophilic monomer used in accordance with the present disclosure may be attached covalently to at least one support such as polymers, oligomers, and dendrimers. The polymer or oligomer may have a structure chosen from linear, branched, comb, and block structures. The distribution of the monomers of the disclosure over the polymeric, oligomeric, or dendritic structure may be chosen from random, terminal, and block distributions.

The at least one electrophilic monomer may be present in the composition in an amount ranging from 0.1% to 80% by weight, for example, from 1% to 50% by weight relative to the total weight of the composition.

The at least one electrophilic monomer may be synthesized according to the known methods described in the literature. For example, the monomers from the cyanoacrylates class may be synthesized according to the teaching of U.S. Pat. Nos. 3,527,224, 3,591,767, 3,667,472, 3,995,641, 4,035,334, and 4,650,826.

According to at least one embodiment of the present disclosure, the at least one electrophilic monomer may be chosen from monomers capable of polymerizing by anionic polymerization in the presence of at least one nucleophile.

As used herein, the term “anionic polymerization” refers to the mechanism defined in J. March, Advanced Organic Chemistry, 3rd ed., pages 151 to 161.

The at least one nucleophile capable of initiating the anionic polymerization may include systems known in the art which are capable of generating a carbanion upon contact with an electrophile, such as the hydroxide ions present in water. As used herein, the term “carbanion” refers to the chemical species defined in J. March, Advanced Organic Chemistry, 3rd ed., page 141.

The at least one nucleophile may be applied to the keratin fibers independently of the composition disclosed herein. It may also be added to the composition disclosed herein at the time of use. In the latter case, the composition in accordance with the present disclosure further comprises at least one nucleophile.

According to at least one embodiment of the disclosure, the at least one nucleophile may be selected from molecular compounds, oligomers, dendrimers, and polymers which carry at least one nucleophilic function selected from R₂N⁻, NH₂⁻, Ph₃C⁻, R₃C⁻, PhNH⁻, pyridine, ArS⁻, R—C≡C⁻, RS⁻, SH⁻, RO⁻, R2NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻ and H₂O, wherein Ph is chosen from phenyl groups, Ar is chosen from aryl groups, and R is chosen from C₁-C₁₀ alkyl groups.

In at least on embodiment, the nucleophile may be water.

According to at least one embodiment of the present disclosure the monomers may be chosen from monomers capable of undergoing polymerization on keratin fibers under cosmetically acceptable conditions. In at least one embodiment, the polymerization of the monomer may take place at a temperature less than or equal to 80° C., for example, at a temperature ranging from 10 to 80° C., or from 20 to 80° C., which does not prevent the application from being completed by heat-treating, for example, drying under a hood, blow-drying, or the passage of a flat iron and/or curling tongs over the keratin fibers.

Cosmetically Acceptable Medium

In accordance with at least one embodiment of the present disclosure, the cosmetically acceptable medium may be an anhydrous, non-hygroscopic medium. As used herein, the term “anhydrous medium” means a medium comprising less than 1% of water.

The cosmetically acceptable medium may be chosen, for example, from:
aromatic alcohols such as benzyl alcohol;
fatty alcohols;
modified or unmodified polyols such as glycerol, glycol, propylene glycol, dipropylene glycol, butylene glycol, and butyl diglycol;
volatile or non-volatile silicones such as cyclopentasiloxane, cyclohexasiloxane, and polydimethylsiloxanes, modified or unmodified with phenyl, siloxy, silanol, amine, imine, fluoroalkyl, carboxyl, betaine, and/or quaternary ammonium functions;
mineral oils, organic oils, and vegetable oils;
oxyethylenated or non-oxyethylenated waxes, paraffins, and alkanes, for example, C₅-C₁₀ alkanes;
fatty acids, fatty amides, and fatty esters, for example fatty alcohol salicylates and fatty alcohol benzoates; and
mixtures thereof.

The cosmetically acceptable medium may also be present in the form of a direct or inverse emulsion and/or may be encapsulated, the electrophilic monomers being held in an anhydrous medium up until the time of use. The disperse or continuous phase of the emulsion may comprise an entity chosen from water, C₁-C₄ aliphatic alcohols, silicones, and mixtures thereof. The capsules or microcapsules containing the composition may be dispersed in an entity chosen from anhydrous mediums as defined above, water, C₁-C₄ aliphatic alcohols, and mixtures thereof.

Optional Additives

The composition in accordance with the present disclosure may further comprise at least one polymerization inhibitor. In at least one embodiment, the at least one polymerization inhibitor may be chosen from anionic and/or free-radical polymerization inhibitors.

Examples of anionic and/or free-radical polymerization inhibitors include, but are not limited to, sulphur dioxide; nitric oxide; organic acids, such as a sulphonic acid, phosphoric acid, and acetic acid; lactone; boron trifluoride; hydroquinone and its derivatives, such as hydroquinone monoethyl ether; tert-butyl hydroquinone; benzoquinone and its derivatives, such as duroquinone; catechol and its derivatives, such as t-butylcatechol and methoxycatechol; anisole and its derivatives, such as methoxyanisole or hydroxyanisole; pyrogallol and its derivatives; p-methoxyphenol; hydroxybutyltoluene; alkyl sulphates; alkyl sulphites; alkyl sulphones; alkyl sulphoxides; alkyl sulphides; mercaptans; 3-sulpholene; and mixtures thereof. The alkyl groups may be chosen from groups comprising from 1 to 6 carbon atoms.

The at least one polymerization inhibitor may be present in the composition in an amount ranging from 0.01% to 10% by weight, for example, from 0.05% and 5% by weight relative to the total weight of the composition.

The oxidizing composition disclosed herein may further comprise at least one thickening polymer which does not exhibit any reactivity with the at least one electrophilic monomer.

Examples of thickening polymers which do not exhibit any reactivity with the at least one electrophilic monomer include, but are not limited to, polymethyl methacrylate (PMMA) and cyanoacrylate-based copolymers, described, for example, in U.S. Pat. No. 6,224,622.

The at least one thickening polymer may be present in the composition in an amount ranging from 0.1% to 50%, for example, from 0.5% and 25%. The at least one thickening polymer may be used, among other things, to modulate the rate of polymerization of the at least one electrophilic monomer.

The composition in accordance with the present disclosure may also contain at least one additional compound conventionally used in cosmetology. The at least one additional compound may be chosen from reducing agents, fatty substances, unmodified or organically modified silicones, thickeners other than the thickening polymers defined above, cationic polymers, anionic polymers, neutral polymers, amphoteric polymers, softeners, antifoams, moisturizers, emollients, alkalifying agents, antioxidants, free-radical scavengers, chelating agents, anti-dandruff agents, seborrhoea regulators, soothing agents, plasticizers, sunscreens, direct dyes (for example, natural or non-natural direct dyes), oxidation dyes (for example, oxidation bases and/or couplers), pigments, mineral fillers, clays, colloidal minerals, nacres, perfumes, peptizers, preservatives, anionic surfactants, cationic surfactants, amphoteric surfactants, zwitterionic surfactants, non-ionic surfactants, fixative or non-fixative polymers, conditioning polymers, hydrolysed or non-hydrolysed proteins, enzymes, amino acids, oligopeptides, peptides, vitamins, saccharides, oligosaccharides, hydrolysed or non-hydrolysed, modified or unmodified polysaccharides, polyamino acids, branched or unbranched fatty alcohols, animal oils, vegetable oils, mineral oils, ceramides, pseudoceramides, hydroxylated organic acids, polyisobutenes, poly(α-olefins), fatty esters, anionic polymers in dissolved or dispersed form, and non-ionic polymers in dissolved or dispersed form.

The skilled person will ensure that the at least one optional additional compound is selected such that the advantageous properties of the composition in accordance with the present disclosure are not, or are not substantially, adversely affected by the at least one additional compound.

The composition in accordance with the invention may be present in a variety of forms, chosen, for example, from lotions, sprays, and mousses, and may be applied in the form of a shampoo or conditioner.

The composition in accordance with the present disclosure may also contain a propellant. The propellant may comprise a compressed or liquefied gas customarily employed for preparing aerosol compositions. In at least one embodiment, the compressed or liquefied gas is chosen from air, carbon dioxide, compressed nitrogen, a soluble gas such as dimethyl ether, halogenated hydrocarbons such as hydrofluorocarbons, non-halogenated hydrocarbons, and mixtures thereof.

Bleaching and/or Permanent Reshaping Methods

Disclosed herein is a method for bleaching and/or permanently reshaping keratin fibers comprising applying to the keratin fibers an oxidizing composition comprising, in a cosmetically acceptable medium, at least one electrophilic monomer and at least one oxidant, in the presence of at least one nucleophile.

The at least one oxidant may be chosen, for example, from:
hydrogen peroxide and agents capable of producing hydrogen peroxide by hydrolysis, such as urea peroxide;
alkali metal or ammonium persulphates, perborates, and percarbonates;
alkali metal bromates;
magnesium monoperoxyphthalate;
nitrites, for example, alkali metal, alkaline earth metal, and ammonium nitrites, such as sodium nitrite and ammonium nitrite, and organic nitrite derivatives;
periodic acid and its water-soluble salts, for example, the lithium, sodium, potassium, rubidium, caesium, magnesium, calcium, strontium, manganese, iron, copper, zinc, and aluminium salts. In at least one embodiment, the periodic acid salts are sodium and potassium salts;
alkali metal ferricyanides, for example, potassium ferricyanide;
silver oxide;
Fenton's reagent;
lead(IV) oxide;
metal anions selected from permanganates and dichromates, for example, potassium permanganate and sodium dichromate;
metal salts of groups III to VIII of the Periodic Table such as manganese, cobalt, iron, copper or silver salts, for instance, manganese sulphate, manganese lactate, cobalt chloride, ferric chloride, cupric chloride, ammoniacal silver nitrate, and copper salts. These metal salts may be in the free state or adsorbed;
rare earth salts chosen from the salts of lanthanides, salts of cerium Ce³⁺ or Ce⁴⁺, salts of lanthanum La³⁺, salts of europium Eu²⁺ or Eu³⁺, salts of gadolinium Gd³⁺, salts of ytterbium Yb²⁺ or Yb³⁺, and salts of dysprosium Dy³⁺, for example, sulphates, chlorides or nitrates. In at least one embodiment, the rare earth salts are sulphate or chloride salts of cerium Ce³⁺ or Ce⁴⁺;
sodium hypochlorite; and
benzoquinone.

According to at least one embodiment of the present disclosure, the oxidizing composition is applied in at least two steps, one of the steps comprising applying to the keratin fibers a composition comprising the at least one electrophilic monomer and the other step comprising applying to the keratin fibers a composition comprising the at least one oxidant, the order of the steps being arbitrary.

The at least one nucleophile may be pure, in solution, in the form of an emulsion, or may be encapsulated. In one embodiment, the at least one nucleophile may be present in the composition comprising the at least one oxidant. In another embodiment, the at least one nucleophile may be applied to the keratin fibers separately. In that case it is possible, for example, to impregnate the keratin fibers beforehand by means of applying the nucleophile.

When the nucleophile is water, the keratin fibers may be wet beforehand using an aqueous solution whose pH has been adjusted by means of acidifying and/or alkalifying agents.

Acidifying agents may include, for example, mineral or organic acids such as hydrochloric acid, orthophosphoric acid, sulphuric acid, carboxylic acids such as acetic acid, tartaric acid, citric acid, lactic acid, and sulphonic acids.

Alkalifying agents may include, but are not limited to, ammonia, alkali metal carbonates, alkanolamines such as mono-, di-, and triethanolamines and their derivatives, sodium hydroxide, potassium hydroxide, and the compounds of formula (VII) below: 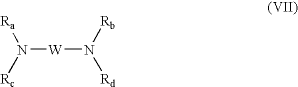
in which W is a propylene residue optionally substituted by at least one entity chosen from hydroxyl groups and C₁-C₄ alkyl radicals; and Rₐ, R_{b}, R_{c}, and R_{d}, which may be identical or different, are chosen from hydrogen, C₁-C₄ alkyl radicals, and C₁-C₄ hydroxyalkyl radicals.

According to at least one embodiment of the present disclosure, the nucleophilicity of the keratin fibers may be enhanced by chemical conversion of the keratin material. As an example, at least one keratin reducing agent may be applied to the keratin fibers in order to reduce the disulphide bridges, of which the keratin is partly composed, to thiols. In one embodiment, the at least one reducing agent may be applied to the keratin fibers before applying the composition comprising the at least one oxidant.

Useful keratin reducing agents may include, but are not limited to:
anhydrous sodium thiosulphate;
powdered sodium metabisulphite;
thiourea;
ammonium sulphite;
thioglycolic acid;
thiolactic acid;
ammonium thiolactate;
glycerol monothioglycolate;
ammonium thioglycolate;
thioglycerol;
2,5-dihydroxybenzoic acid;
diammonium dithioglycolate;
strontium thioglycolate;
calcium thioglycolate;
zinc formaldehyde-sulfoxylate;
isooctyl thioglycolate;
dl-cysteine; and
monoethanolamine thioglycolate.

In order to improve, among other things, the adhesion of the polycyanoacrylate formed in situ, the keratin fibers may be pretreated with polymers of any type. Optionally, a hair treatment may be performed before applying the oxidizing composition to the keratin fibers, such as a direct or oxidation dyeing, a perming, and/or a relaxing treatment.

The application of the compositions to the keratin fibers may or may not be followed by a rinsing and/or drying operation. Drying may be carried out using a hood, a hairdryer, and/or straightening tongs.

The composition may also contain various additional compounds as defined above.

According to at least one embodiment of the present disclosure, the bleaching and/or permanent reshaping method may comprise multiple superpositions of applications.

Also disclosed herein is a kit for bleaching and/or permanently reshaping keratin fibers, comprising a first composition comprising at least one electrophilic monomer and optionally at least one polymerization inhibitor and a second composition comprising at least one oxidant.

The at least one electrophilic monomer may be present in the first composition in an amount ranging from 0.05% to 30% by weight, for example, from 0.01% to 50% by weight, or from 0.1% to 20% by weight.

According to one embodiment of the present disclosure, the second composition may further comprise at least one nucleophile.

According to another embodiment of the present disclosure, the kit may further comprise a third composition comprising at least one nucleophile.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements.

By way of non-limiting illustration, concrete examples of certain embodiments of the present disclosure are given below. The percentages are expressed on a weight basis of active material, unless otherwise indicated.

### EXAMPLES

Examples 1 and 2 below were performed using the following compounds:
Monomer: n-octyl cyanoacrylate stabilized with 1% of phosphoric acid.
Cosmetic Medium:
50% of a 14.7/85.3 α,ω-codihydroxypolydimethyl-siloxane/cyclopentadimethylsiloxane mixture sold by Dow Corning under the name DC 1501 Fluid;
50% of cyclopentadimethylsiloxane sold by Dow Corning under the name DC245 Fluid.

### Example 1

A composition for hair bleaching was prepared, comprising, in the cosmetic medium, 4.5% by weight of potassium persulphate, 50% by weight of hydrogen peroxide solution (6% by weight), 3% by weight of aqueous ammonia (containing 20.5% NH₃), and 10% by weight of monomer.

Five grams of the composition thus obtained were applied to a 1 g lock of natural chestnut hair for 30 minutes at ambient temperature.

After a 30-minute waiting period, the lock was rinsed with clean water and dried with a hairdryer.

The lock obtained was bleached and exhibited a very cosmetic feel.

### Example 2

An aqueous composition was prepared with 50% by weight of 20-volume hydrogen peroxide solution and 10% by weight of aqueous ammonia (containing 40% NH₃ by weight in water).

Five grams of this aqueous composition were applied to a 1 g lock of chestnut hair for 30 minutes at ambient temperature.

After a 30-minute waiting period, the lock was rinsed with clean water.

0.5 g of a solution of the cosmetic medium containing 10% by weight of monomer was applied to the lock.

After a 10-minute waiting time at ambient temperature the lock was dried for 2 minutes with a hairdryer.

The lock obtained was bleached and had a very cosmetic feel.

### Examples 3 to 8

The following hair-bleaching compositions were prepared:

| | Example | | | | | | | | | | | |
|---|---|---|---|---|---|---|---|---|---|---|---|---|
| | 3 | | 4 | | 5 | | 6 | | 7 | | 8 | |
| Methylheptyl cyanoacrylate | 10 | g | — | | — | | — | | 9 | g | 7 | g |
| sold by Chemence | | | | | | | | | | | | |
| Ethoxyethyl cyanoacrylate | — | | 10 | g | — | | — | | — | | — | |
| EO 460 sold by Tong Shen | | | | | | | | | | | | |
| Butyl cyanoacrylate B 60 | — | | — | | 10 | g | — | | — | | 3 | g |
| sold by Tong Shen | | | | | | | | | | | | |
| Ethylhexyl cyanoacrylate | — | | — | | — | | 10 | g | 1 | g | — | |
| O 60 sold by Tong Shen | | | | | | | | | | | | |
| DC 245 Fluid | 33.75 | g | 33.75 | g | 33.75 | g | 33.75 | g | 33.75 | g | 33.75 | g |
| DC 1501 Fluid | 33.75 | g | 33.75 | g | 33.75 | g | 33.75 | g | 33.75 | g | 33.75 | g |
| Potassium persulphate | 4.5 | g | 4.5 | g | 4.5 | g | 4.5 | g | 4.5 | g | 4.5 | g |
| Hydrogen peroxide solution | 50 | g | 50 | g | 50 | g | 50 | g | 50 | g | 50 | g |
| containing 6% H₂O₂ | | | | | | | | | | | | |
| Aqueous ammonia | 3 | g | 3 | g | 3 | g | 3 | g | 3 | g | 3 | g |
| containing 20.5% NH₃ | | | | | | | | | | | | |

Each of the compositions was applied to a 1 g lock of natural chestnut hair for 30 minutes at ambient temperature. The locks were subsequently rinsed and dried.

The locks obtained were bleached and their cosmetic properties were unaltered.

### Example 9

The following compositions were prepared:

Composition A: composition comprising an electrophilic monomer

| | |
|---|---|
| Methylheptyl cyanoacrylate sold by Chemence | 10 g |
| DC 245 Fluid | 45 g |
| DC 1501 Fluid | 45 g |

Composition B: bleaching composition

| | |
|---|---|
| Hydrogen peroxide solution containing 6% H₂O₂ | 50 g |
| Aqueous ammonia containing 20.5% NH₃ | 10 g |

Composition A was applied to a 1 g lock of natural chestnut hair, which was clean and wet, for 15 minutes at ambient temperature, followed by drying. Composition B was subsequently applied to the lock for 30 minutes at ambient temperature, followed by rinsing and drying.

The lock obtained was bleached and its cosmetic properties were unaltered.

### Example 10

The following compositions were prepared:

Composition C: composition comprising a reducing agent

| | |
|---|---|
| Thioglycolic acid | 9 g |
| Aqueous ammonia | qs pH 9 |
| Water | qs 100 |

Composition D: composition comprising an oxidant

| | |
|---|---|
| Hydrogen peroxide solution containing 50% H₂O₂ | 4.8 g |
| Citric acid | qs pH 3 |
| Water | qs 100 |

Composition E: composition comprising an electrophilic monomer

| | |
|---|---|
| Methylheptyl cyanoacrylate sold by Chemence | 10 g |
| DC 245 Fluid | 45 g |
| DC 1501 Fluid | 45 g |

Composition C was applied to a 1 g lock of natural chestnut hair, which was clean and wet and had been wound on a roller, for 15 minutes at ambient temperature, followed by rinsing. Subsequently Composition D was applied to the lock for 15 minutes at ambient temperature, followed by rinsing. Subsequently Composition E was applied to the lock for 15 minutes at ambient temperature. The lock was then dried and unwound from the roller.

The lock obtained was shaped without any adverse effect on its cosmetic properties.

### Example 11

The following compositions were prepared:

Composition F: composition comprising an electrophilic monomer

| | |
|---|---|
| Methylheptyl cyanoacrylate sold by Chemence | 10 g |
| DC 245 Fluid | 45 g |
| DC 1501 Fluid | 45 g |

Composition G: composition comprising a reducing agent

| | |
|---|---|
| Thioglycolic acid | 9 g |
| Aqueous ammonia | qs pH 9 |
| Water | qs 100 |

Composition H: composition comprising an oxidant

| | |
|---|---|
| Hydrogen peroxide solution containing 50% H₂O₂ | 4.8 g |
| Citric acid | qs pH 3 |
| Water | qs 100 |

Composition F was applied to a 1 g lock of natural chestnut hair, which was clean and wet, for 15 minutes at ambient temperature. The lock was subsequently dried, wetted again, and wound up on a roller. Subsequently Composition G was applied to the lock for 15 minutes at ambient temperature, followed by rinsing. Subsequently Composition H was applied to the lock for 15 minutes at ambient temperature. The lock was then rinsed and unwound from the roller.

The lock was shaped without any adverse effect on its cosmetic properties.

What is claimed is:

1. An oxidizing composition comprising, in a cosmetically acceptable medium:
at least one electrophilic monomer chosen from the compounds of formula (I): 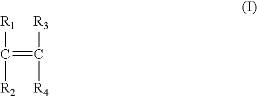
in which:
R₁ and R₂, are, independently of one another, minimally or non-electron-withdrawing groups; and
R₃ and R₄, are, independently of one another, electron-withdrawing groups; and
at least one non-benzoquinone oxidant.

2. The composition of claim 1, wherein the at least one oxidant is chosen from:
hydrogen peroxide and agents capable of producing hydrogen peroxide by hydrolysis;
alkali metal or ammonium persulphates, perborates, and percarbonates;
alkali metal bromates;
magnesium monoperoxyphthalate;
nitrites;
periodic acid and its water-soluble salts;
alkali metal ferricyanides;
silver oxide;
Fenton's reagent;
lead(IV) oxide;
metal anions chosen from permanganates and dichromates;
metal salts from groups III to VIII of the Periodic Table;
rare earth salts chosen from salts of lanthanides, salts of cerium Ce³⁺ or Ce⁴⁺, salts of lanthanum La³⁺, salts of europium Eu²⁺ or Eu³⁺, salts of gadolinium Gd³⁺, salts of ytterbium Yb²⁺ or Yb³⁺, and salts of dysprosium Dy³⁺; and
sodium hypochlorite.

3. The composition of claim 2, wherein the at least one oxidant is chosen from hydrogen peroxide and agents capable of producing hydrogen peroxide by hydrolysis.

4. The composition of claim 3, wherein the agents capable of producing hydrogen peroxide by hydrolysis comprise urea peroxide.

5. The composition of claim 3, wherein the at least one oxidant is chosen from persulphates.

6. The composition of claim 1, wherein the at least one oxidant is present in the composition in an amount ranging from 0.05% to 40% by weight relative to the total weight of the composition.

7. The composition of claim 1, wherein R₁ and R₂, are independently chosen from:
hydrogen;
saturated or unsaturated, linear, branched, or cyclic hydrocarbon-based groups comprising from 1 to 20 carbon atoms, and optionally comprising at least one atom chosen from nitrogen, oxygen, and sulphur, and optionally substituted by at least one group chosen from —OR, —COOR, —COR, —SH, —SR, —OH, and halogen atoms;
modified or unmodified polyorganosiloxane residues; and
polyoxyalkylene groups;
wherein R is chosen from saturated or unsaturated linear, branched, or cyclic hydrocarbon-based groups comprising from 1 to 20 carbon atoms, and optionally comprising at least one atom chosen from nitrogen, oxygen, and sulphur, and optionally substituted with at least one group chosen from —OR′, —COOR′, —COR′, —SH, —SR′, —OH, halogen atoms, and polymer residues; and
wherein R′ is chosen from C₁-C₁₀ alkyl radicals.

8. The composition of claim 1, wherein R₃ and R₄ are independently chosen from —N(R)₃+, —S(R)₂+, —SH₂+, —NH₃+, —NO₂, —SO₂R, —C—N, —COOH, —COOR, —COSR, —CONH₂, —CONHR, —F, —Cl, —Br, —I, —OR, —COR, —SH, —SR, and —OH, linear or branched alkenyl groups, linear or branched alkynyl groups, C₁-C₄ mono- or polyfluoroalkyl groups, aryl groups, and aryloxy groups;
wherein R is chosen from saturated or unsaturated linear, branched, or cyclic hydrocarbon-based groups ccomprising from 1 to 20 carbon atoms, and optionally comprising at least one atom chosen from nitrogen, oxygen, or sulphur, and optionally substituted with at least one group chosen from —OR′, —COOR′, —COR′, —SH, —SR′, —OH, halogen atoms, and polymer residues;
wherein R′ is chosen from C₁-C₁₀ alkyl radicals.

9. The composition of claim 1, wherein the at least one electrophilic monomer is chosen from:
benzylidenemalononitrile (A), 2-(4-chlorobenzylidene)malononitrile (A1), ethyl 2-cyano-3-phenylacrylate (B) and ethyl 2-cyano-3-(4-chlorophenyl)acrylate (B1): 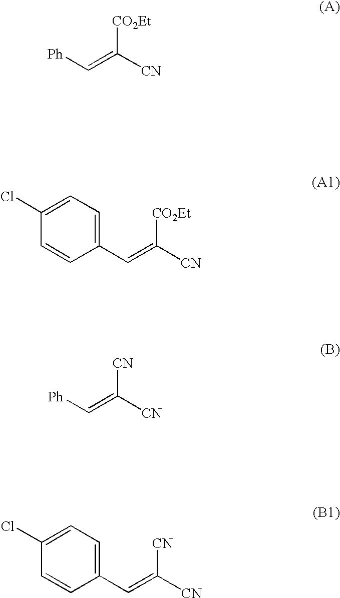
diethyl 2-methylenemalonate (C): 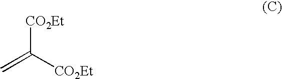
ethyl 2-ethoxycarbonylmethyleneoxycarbonylacrylate (D): 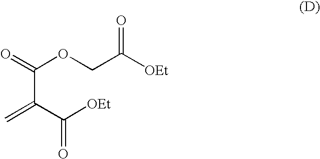
methyl α-(methylsulphonyl)acrylate derivatives (K), ethyl α-(methylsulphonyl)acrylate derivatives (L), methyl α-(tert-butylsulphonyl)acrylate derivatives (M), tert-butyl α-(methylsulphonyl)acrylate derivatives (N), tert-butyl α-(tert-butylsulphonyl)acrylate derivatives (O): 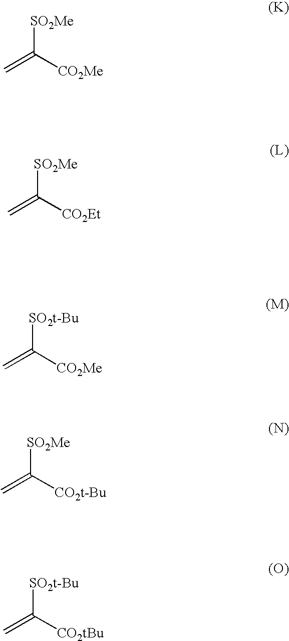
1,1-bis(methylsulphonyl)ethylene derivatives (P), 1-acetyl-1-methylsulphonylethylene derivatives (Q), methyl α-(methylsulphonyl)vinylsulphonate derivatives (R), and α-methylsulphonylacrylonitrile derivatives (S):

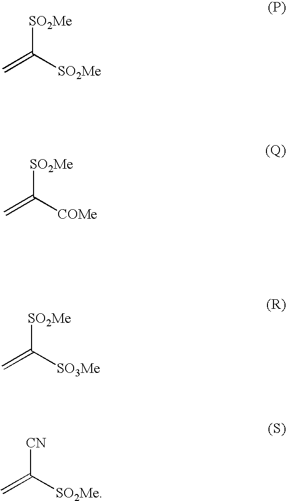

10. The composition of claim 1, wherein the at least one electrophilic monomer is chosen from the monomers of the cyanoacrylate class of formula (II): 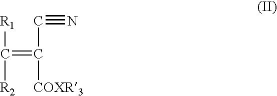
in which:
X is chosen from NH, S, and O;
R′₃ is chosen from hydrogen and the radical R;
R₁ and R₂, are independently chosen from:
hydrogen;
saturated or unsaturated, linear, branched, or cyclic hydrocarbon-based groups comprising from 1 to 20 carbon atoms, and optionally comprising at least one atom chosen from nitrogen, oxygen, and sulphur, and optionally substituted by at least one group chosen from —OR, —COOR, —COR, —SH, —SR, —OH, and halogen atoms;
modified or unmodified polyorganosiloxane residues; and
polyoxyalkylene groups;
wherein R is chosen from saturated or unsaturated linear, branched, or cyclic hydrocarbon-based groups comprising from 1 to 20 carbon atoms, and optionally comprising at least one atom chosen from nitrogen, oxygen, and sulphur, and optionally substituted with at least one group chosen from —OR′, —COOR′, —COR′, —SH, —SR′, —OH, and halogen atoms, and polymer residues; and
wherein R′ is chosen from C₁-C₁₀ alkyl radicals.

11. The composition of claim 1, wherein R₁ and R₂ are hydrogen atoms.

12. The composition of claim 10, wherein X is O.

13. The composition of claim 10, wherein R′₃ is chosen from C₆-C₁₀ alkyl radicals.

14. The composition of claim 1, wherein the at least one electrophilic monomer is chosen from the monomers of the alkyl or alkoxyalkyl 2-cyanoacrylate class, of formula (V): 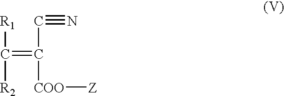
in which:
Z is chosen from C₁-C₁₀ alkyl radicals and C₁-C₄-alkoxy-C₁-C₁₀ alkyl radicals; and
R₁ and R₂, are independently chosen from:
hydrogen;
saturated or unsaturated, linear, branched, or cyclic hydrocarbon-based groups comprising from 1 to 20 carbon atoms, and optionally comprising at least one atom chosen from nitrogen, oxygen, and sulphur, and optionally substituted by at least one group chosen from —OR, —COOR, —COR, —SH, —SR, —OH, and halogen atoms;
modified or unmodified polyorganosiloxane residues; and
polyoxyalkylene groups;
wherein R is chosen from saturated or unsaturated linear, branched, or cyclic hydrocarbon-based groups comprising from 1 to 20 carbon atoms, and optionally comprising at least one atom chosen from nitrogen, oxygen, and sulphur, and optionally substituted with at least one group chosen from —OR′, —COOR′, —COR′, —SH, —SR′, —OH, and halogen atoms, and polymer residues; and
wherein R′is chosen from C₁-C₁₀ alkyl radicals.

15. The composition of claim 14, wherein Z is chosen from C₆-C₁₀ alkyl radicals.

16. The composition of claim 14, wherein R₁ and R₂ are hydrogen atoms.

17. The composition of claim 1, wherein the at least one electrophilic monomer is chosen from the compounds of formula (VI) and mixtures thereof: 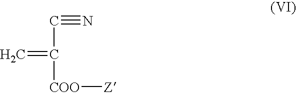
in which Z′ is chosen from
—(CH₂)₇—CH₃;
—CH(CH₃)—(CH₂)₅—CH₃;
—CH₂—CH(C₂H₅)—(CH₂)₃—CH₃;
—(CH₂)₅—CH(CH₃)—CH₃; and
—(CH₂)₄—CH(C₂H₅)—CH₃.

18. The composition of claim 1, wherein the at least one electrophilic monomer is present in the composition in an amount ranging from 0.1% to 80% by weight relative to the total weight of the composition.

19. The composition of claim 1, wherein the cosmetically acceptable medium is anhydrous.

20. The composition of claim 19, wherein the cosmetically acceptable medium is chosen from:
aromatic alcohols;
fatty alcohols;
modified polyols and unmodified polyols;
volatile silicones and non-volatile silicones;
mineral oils, organic oils, and vegetable oils;
oxyethylenated or non-oxyethylenated waxes, paraffins, and alkanes;
fatty acids, fatty amides, and fatty esters; and
mixtures thereof.

21. The composition of claim 1, further comprising at least one nucleophile.

22. The composition of claim 21, wherein the at least one nucleophile is water.

23. A method for bleaching and/or permanently reshaping keratin fibers, comprising applying to the keratin fibers an oxidizing composition comprising, in a cosmetically acceptable medium, at least one electrophilic monomer and at least one non-benzoquinone oxidant in the presence of at least one nucleophile, wherein
the at least one electrophilic monomer chosen from the compounds of formula (I): 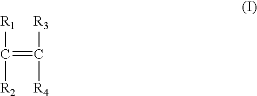
in which:
R₁ and R₂, are, independently of one another, minimally or non-electron-withdrawing groups; and
R₃ and R₄, are, independently of one another, electron-withdrawing groups.

24. The method of claim 23, wherein the at least one oxidant is chosen from:
hydrogen peroxide and agents capable of producing hydrogen peroxide by hydrolysis;
alkali metal or ammonium persulphates, perborates, and percarbonates;
alkali metal bromates;
magnesium monoperoxyphthalate;
nitrites;
periodic acid and its water-soluble salts;
alkali metal ferricyanides;
silver oxide;
Fenton's reagent;
lead(IV) oxide;
metal anions chosen from permanganates and dichromates;
metal salts from groups III to VIII of the Periodic Table;
rare earth salts chosen from the salts of lanthanides, salts of cerium Ce³⁺ or Ce⁴⁺, salts of lanthanum La³⁺, salts of europium Eu²⁺ or Eu³⁺, salts of gadolinium Gd³⁺, salts of ytterbium Yb²⁺ or Yb³⁺, and salts of dysprosium Dy³⁺; and
sodium hypochlorite.

25. The method of claim 23, wherein the oxidizing composition is applied in at least two steps, wherein the first step comprises applying to the keratin fibers a first composition comprising the at least one electrophilic monomer and the second step comprises applying to the keratin fibers a second composition comprising the at least one oxidant, the order of the steps being arbitrary.

26. The method of claim 23, wherein the nucleophile is applied to the keratin fibers separately from the oxidizing composition.

27. The method of claim 25, wherein the second composition further comprises at least one nucleophile.

28. The method of claim 23, further comprising applying to the keratin fibers at least one keratin reducing agent.

29. The method of claim 28, wherein the at least one reducing agent is applied to the keratin fibers before the ozidizing composition.

30. A kit for bleaching and/or permanently reshaping keratin fibers, comprising a first composition comprising at least one electrophilic monomer and at least one polymerization inhibitor and a second composition comprising at least one non-benzoquinone oxidant, wherein
the at least one electrophilic monomer chosen from the compounds of formula (I): 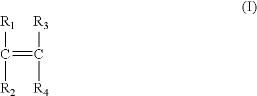
in which:
R₁ and R₂, are, independently of one another, minimally or non-electron-withdrawing groups; and
R₃ and R₄, are, independently of one another, electron-withdrawing groups.

31. The kit of claim 30, wherein the second composition further comprises at least one nucleophile.

32. The kit of claim 31, wherein the at least one nucleophile is water.

33. The kit of claim 30, further comprising a third composition comprising at least one nucleophile.
